SEQUENCE LISTING (7 sequences)

SEQ ID NO: 1 - DNA, 1032 bp, Bacillus paralicheniformis
  misc_feature 1..1032: note = Heat-inducible transcription repressor HrcA nucleotide sequence in B. paralicheniformis CHCC3809
  source 1..1032: mol_type = genomic DNA
atgttaacaaatcgtcagctgttaatcttgcaggttatcgtcaacgatttcattcgttcagctcagccggtaggatcaagaacgctttccaaaaaggaagatatcacattcagctcagcaacgatcagaaacgaaatggctgacttggaagagctcggttttattgaaaaaacccactcatcttcaggccggattccttctgaaaaaggctatcgctattatgtcgatcatctgctttcacccggaaagctgtcaaaaacggacttgaacattattcattcggttttcaaagaaaaaatctttgaactcgaaaaagcggtgcagaagtcggctcaagtgctgtctgatctgacaaattatacatcgattgtcctcggtccgagactgagcgaaaatcatctcaaacagatccagattgtgccgattcagcctaagaaggccgttgccattctagtaacgaataccggccatgtcgagaataaaacgatcaactttccggcggaggtcaatctttccgatctcgaaaagctggtgaatatattaaatgaacgccttagaggcgtgccgatctcagagctgaaagacaggattttcaaagaggtcgtcatcttcttaaagtcgcatatccaaaattacgatacgattttacacgggctcggcgcaacgctggattcatctgttcaaaccgaccggctgtttttcggcggcaagattaatatgctgaatcagcccgaatttcacgatattgacagagtgaaatcgctattgtcgctcattgagaaagaacaggagcttctccggctctttcagtcgactgagtccggaattaccattaaaatcggcaaggaaaacgactatgaagaaatggaaaactgcagcctgattaccgcgacatacacggtcggttcaaaacagatcggctccatcgcggtcatcgggccgacgcgcatggactactcccgcgtcgtcggtttgcttcagcacgtatcatctgacttgtcaaaagcgttgacaagtttgtatgatgggtaa

SEQ ID NO: 2 - protein, 343 aa, Bacillus paralicheniformis
MLTNRQLLILQVIVNDFIRSAQPVGSRTLSKKEDITFSSATIRNEMADLEELGFIEKTHSSSGRIPSEKGYRYYVDHLLSPGKLSKTDLNIIHSVFKEKIFELEKAVQKSAQVLSDLTNYTSIVLGPRLSENHLKQIQIVPIQPKKAVAILVTNTGHVENKTINFPAEVNLSDLEKLVNILNERLRGVPISELKDRIFKEVVIFLKSHIQNYDTILHGLGATLDSSVQTDRLFFGGKINMLNQPEFHDIDRVKSLLSLIEKEQELLRLFQSTESGITIKIGKENDYEEMENCSLITATYTVGSKQIGSIAVIGPTRMDYSRVVGLLQHVSSDLSKALTSLYDG

SEQ ID NO: 3 - DNA, 1152 bp, Bacillus paralicheniformis
  misc_feature 1..1152: note = BioF region 8-amino-7-oxononanoate synthase (EC_2.3.1.47) nucleotide sequence in B. paralicheniformis CHCC3809
  source 1..1152: mol_type = genomic DNA
gtgcccattgacgaatggctcagcagtcggctcgccaggacgaaggcagccgggctttaccgctcgcttaaaccgcctcaagctgtcgcggaagcgaaacgtacgaatcgggcgtcaaacgattatttgagcctggcgaacgacaaacggctgattcacgccgcagaaacggcgcttcgccgctttggagccgggagcacagggtcaaggctgacgtccggcaataccgaatggcatgaaaaacttgagcggaaaatcgccggttttaagcagacagaagccgctctcttgttttcaagcggatatttggccaatatcggcgtgctttcgtctttgcctgaaaaaggagacgtcatattaagcgatcaattgaaccatgcgagcatcatcgacggctgccgcctttcaaaagctgatacggttgtttaccgccacacggatatgaatgatcttgaagaaaaactccgcaccgcacagagccgtgctcgctgttttattgtgacagacggcgtgttcagcatggacggcacaattgcaccgcttgacgaaatcatgctccttgccaagcggtaccgggcctttgtcatcgttgatgatgcccacgcaacaggggttttgggagatgccggcaggggaacgggcgagtatttcggcgtctccccagatgttgtgatcggcacgttaagtaaagctgtaggcgcggaaggcggcttcgtcgcgggctctaaagcattgatcgattttttgctgaatcatgcgagaacgtttatctttcaaacggctgtcccgcccgcaagctgtgcggcggcatgcagagctttagacatcattaaagacagccgggataaacgccggcttttgcaatcatcggtgaccacgatcaaacggggtctcgaggatatcgggtttacggtcaagggagaagatacgccgatcatacccgttatgatcggagaccctcaaaaagccgttcgatttgcaaacggcctcaaggagaaagggattgaggccccggccatccgcccgccgactgttgcggaaggagagagccgaatcaggctgaccgtcaccgcagatcgcaagttaagagatattgaagctctattagaggggtttaaattagtgggaagagagttgaacttggtgaaatga

SEQ ID NO: 4 - protein, 383 aa, Bacillus paralicheniformis
MPIDEWLSSRLARTKAAGLYRSLKPPQAVAEAKRTNRASNDYLSLANDKRLIHAAETALRRFGAGSTGSRLTSGNTEWHEKLERKIAGFKQTEAALLFSSGYLANIGVLSSLPEKGDVILSDQLNHASIIDGCRLSKADTVVYRHTDMNDLEEKLRTAQSRARCFIVTDGVFSMDGTIAPLDEIMLLAKRYRAFVIVDDAHATGVLGDAGRGTGEYFGVSPDVVIGTLSKAVGAEGGFVAGSKALIDFLLNHARTFIFQTAVPPASCAAACRALDIIKDSRDKRRLLQSSVTTIKRGLEDIGFTVKGEDTPIIPVMIGDPQKAVRFANGLKEKGIEAPAIRPPTVAEGESRIRLTVTADRKLRDIEALLEGFKLVGRELNLVK

SEQ ID NO: 5 - protein, 383 aa, Escherichia coli
MSWQEKINAALDARRAADALRRRYPVAQGAGRWLVADDRQYLNFSSNDYLGLSHHPQIIRAWQQGAEQFGIGSGGSGHVSGYSVVHQALEEELAEWLGYSRALLFISGFAANQAWIAAMMAKEDRIAADRLSHASLLEAASLSPSQLRRFAHNDVTHLARLLASPCPGQQMVVTEGVFSMDGDSAPLAEIQQVTQQHNGWLMVDDAHGTGVIGEQGRGSCWLQKVKPELLVVTFGKGFGVSGAAVLCSSTVADYLLQFARHLIYSTMPPAQAQALRASLAVIRSDEGDARREKLAALITRFRAGVQDLPFTLADSCSAIQPLIVGDNSRALQLAEKLRQQGCWVTAIRPPTVPAGTARLRLTLTAAHEMQDIDRLLEVLHGNG

SEQ ID NO: 6 - protein, 388 aa, Bacillus subtilis
MKIDSWLNERLDRMKEAGVHRNLRSMDGAPVPERNIDGENQTVWSSNNYLGLASDRRLIDAAQTALQQFGTGSSGSRLTTGNSWVHEKLEKKIASFKLTEAALLFSSGYLANVGVLSSLPEKEDVILSDQLNHASMIDGCRLSKADTVVYRHIDMNDLENKLNETQRYKRRFIVTDGVFSMDGTIAPLDQIISLAKRYHAFVVVDDAHATGVLGDSGQGTSEYFGVCPDIVIGTLSKAVGAEGGFAAGSAVFIDFLLNHRTFIFQTAIPPASCAAAHEAFNIIEASREKRQLLFSYISMIRTSLKNMGYVVKGDHTPIIPVVIGDAHKTVLFAEKLQGKGIYAPAIRPPTVAPGESRIRITITSDHSMGDIDHLLQTFHSIGKELHII

SEQ ID NO: 7 - protein, 383 aa, Bacillus paralicheniformis; identical to SEQ ID NO: 4

(19) United States
(12) Patent Application Publication
(10) Pub. No.: US 2026/0240169 A1
(43) Pub. Date: Aug. 20, 2026

(54) Novel Temperature-Optimized Bacilli

(71) Applicant: Chr. Hansen A/S, Hoersholm (DK)

(72) Inventors: Patricia Dominguez CUEVAS, Hoersholm (DK); Raquel Azevedo, Hoersholm (DK); Karin Bjerre, Hoersholm (DK); Iuliana Nita, Hoersholm (DK); Rute Neves, Kobenhavn (DK); Lars Moelbak, Hoersholm (DK)

(73) Assignee: Chr. Hansen A/S, Hoersholm (DK)

(21) Appl. No.: 19/549,111

(22) Filed: Feb. 25, 2026

Related U.S. Application Data
(62) Division of application No. 17/638,152, filed on Aug. 28, 2020, now Pat. No. 12,593,849.

(30) Foreign Application Priority Data
Aug. 29, 2019 (EP) 19194343.0

(51) Int. Cl. A01N 63/22; C07K 14/32

(52) U.S. Cl. CPC A01N 63/22; C07K 14/32

(57) ABSTRACT

The present invention relates to new strains of Bacillus paralicheniformis with improved growth rates and plant growth promoting properties and new strains of Bacillus paralicheniformis with improved growth rates when compared to their parental strains from where they are derived.

### CROSS-REFERENCE TO RELATED APPLICATIONS

This application is a divisional of Ser. No. 17/638,152, filed Feb. 24, 2022, which is a national stage entry of PCT/EP2020/074126, filed Aug. 28, 2020, which claims priority to European patent application no. 19194343.0, filed Aug. 29, 2019. The contents of the aforementioned applications are fully incorporated herein by reference.

### SEQUENCE LISTING

This application contains a Sequence Listing in computer readable form, which is incorporated herein by reference. The name of the file containing the Sequence Listing is SQ.xml, which was created on Feb. 12, 2026, and which contains 12,089 bytes.

### SUMMARY OF THE INVENTION

The present invention relates to a composition comprising Bacilli and Bacillus paralicheniformis with increased growth rates and higher biomass yields at different temperatures, to its use, to a process for its preparation, to the use of Bacilli and Bacillus paralicheniformis with increased growth rates and higher biomass yields at different temperatures for controlling, combating and/or conferring specific resistance to plant pests. Particularly, the invention relates to strains of Bacilli and B. paralicheniformis with altered functionality of the proteins encoded by one or more of the genes BioF and HrcA.

The inventors of present invention have generated strains that show increased growth rates and higher biomass yields at a range of different temperatures.

Further, selected new strains of present invention is shown to induce a slight improvement in plant growth experiments, as compared to the original strain in the model plant organism Arabidopsis thaliana.

Genotypic variations associated with the phenotypic changes are described. Derivative strains have been tested in plant experimental systems and proven to promote increased plant growth as compared to the original strain. Further, two distinct mechanisms explaining the impact of the genetic modifications disclosed and discussed herein.

### FIELD OF THE INVENTION

In the current context of a modern and ecologic society, which is concerned with preserving the environment, biological control is considered an attractive alternative or supplement to conventional methods of control. Biological control is the use of one organism (predator, parasite or pathogen) that attacks another organism which is causing economic damage to crops. This is a very common strategy in agroecological systems, as well as in conventional agriculture which relies on the Integrated Pest Management (IPM).

Although the biological control brings positive effects in the reduction or withdrawal of pesticide use and improving farmers' income, an analysis of the set of experiments worldwide, shows that the results are still concentrated in only a few crops and in select geographies with climates supporting the growth rates of Bacilli and in particular plant growth-promoting rhizobacteria as B. paralicheniformis. There is still much to develop in areas of control of pests and diseases.

There has been a great emphasis on research on biological control with the use of bacteria colonizing the roots of plants, called rhizobacteria. The beneficial rhizobacteria for promoting growth and/or acting in the biological control of plant pathogenic bacteria are called plant growth-promoting rhizobacteria or PGPR.

One of the key factors for successful biological control by PGPR is successful colonization of the habitat e.g. by growth rates and biomass. Hence successful biofilm formation may increase the protective effect of the PGPR.

In summary, cold adapted derivative B. paralicheniformis DSM31110 strains were generated following an adaptive laboratory evolution campaign. 14 improved derivatives were selected, and their genomes sequenced to identify the acquired genotypic changes. Derivative strains were characterized physiologically and tested for performance in plant growth experiments. Based on results herein we propose mechanisms to explain the observed phenotypic differences with the parental strain. In addition, these evolution experiments have contributed with a new Bacillus strain showing improved properties in plant growth promotion.

### STATE OF THE ART

The pressure of society to replace the chemicals with environmentally acceptable products or ecological techniques has encouraged the search for alternative methods to promote plant health. In this context, biological control has been considered one of the alternatives within an integrated approach, in which one seeks to ensure sustainable development of agriculture.

The risks to humans and environments presented by using synthetic pesticides emphasize the need for tools such as biological control in optimizing sustainable agricultural systems.

Based on the idea that improved growth rates and biomass formation at different temperatures may improve the bioprotective effect of Bacilli, the inventors of present invention have selected derivative strains of Bacilli that show an increase in growth rates and biomass formation at different temperatures.

To the best of our knowledge specific genetic features linked to increased growth rates and biomass formation at different temperatures and associated mode of actions have never been described for Bacilli and in particular not for B. paralicheniformis strains.

### BRIEF DESCRIPTION OF THE DRAWINGS

FIG. 1—ALE generation of cold adapted strains: experimental approach. A. ALE experimental approach to select for cold adapted DSM31110 derivatives was followed for 12 weeks. 5-10% dilution to fresh medium of 12 independent overnight cultures was done daily (6 independent cultures were evolved in two different growth media). OD (600 nm) measurements were taken daily and number of generations per week calculated. Samples from each tube were kept frozen as glycerol stocks weekly. Temperature was gradually decreased from 25° C. to 18° C. throughout the ALE progression. B. After 12 weeks of adaptive laboratory evolution, serial dilutions from each culture were plated, and 2×960 independent clones were selected, grown in LB broth, and kept frozen as glycerol stocks in 96-well microtiter plates. Individual clone growth profiles were compared first in a Growth Profiler 960 at 21° C. The 14 faster growers were selected and their growth patterns analyzed in different growth media and temperatures.

FIG. 2—Growth profiles of the selected cold adapted derivatives vs parental strain DSM31110. A. Cold adapted derivatives evolved in MSgg growth medium were grown in MSgg medium at 21° C. and compared with the parental strain DSM31110. B. Cold adapted derivatives evolved in MS-Rex growth medium were grown in MS-Rex medium at 21° C. and compared with the parental strain DSM31110. Maximum biomass yields reached by the different strains were determined.

FIG. 3—Maximum growth rates (μmax) calculated for each derivative strain and parental strain DSM31110. A. μmax values reached in MSgg medium at 21° C. B. μmax values reached in MS-Rex at 21° C.

FIG. 4—Growth profiles for cold adapted derivatives in different growth media at 30° C. temperature. Growth profiles for selected cold adapted derivatives vs parental strain DSM31110. (A-B) Cold adapted derivatives were grown in LB medium at 30° C. and compared with the parental strain DSM31110. (C-D) Cold adapted derivatives were grown in BHI medium at 30° C. and compared with the parental strain DSM31110. X axes correspond to time scale of the experiment in hours. Maximum yields reached by the different strains was determined. A-C panels correspond to derivatives evolved in MSgg growth medium. B-D panels correspond to derivatives evolved in MS-Rex growth medium.

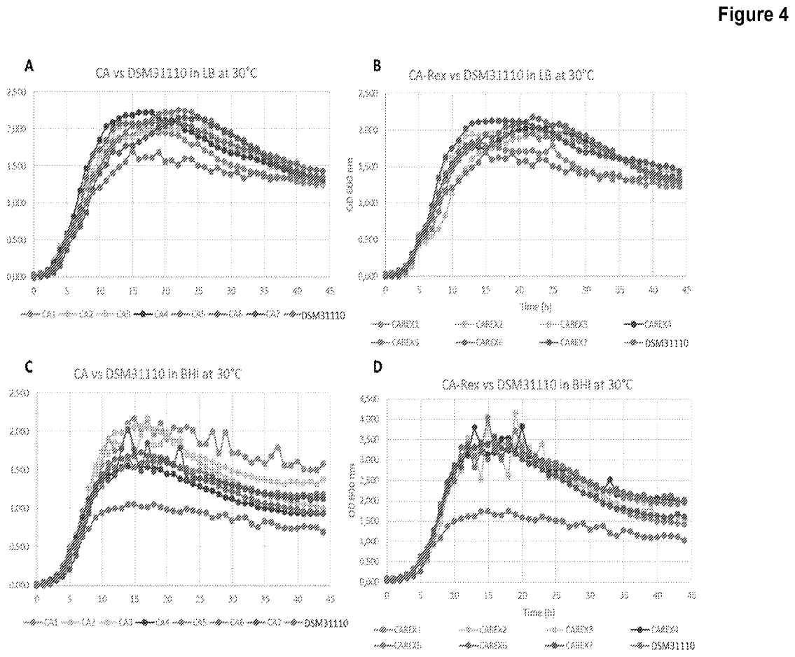

FIG. 5—Plant growth promotion experiments. Plant growth and fitness promotion quantification results from DSM31110 and 14 cold-adapted improved derivative strains CA1-CA7, and CAREX1-CAREX7 corresponding to CHCC32528-CHCC32529-CHCC36494-CHCC36497, and CHCC36751-36757, respectively. Results correspond to experiments done in an in vitro agar system. Left panel corresponds to the total leaf area measurements average calculated per plant (48 plants per strain). Central panel corresponds to the average fresh weight per plant. Right panel corresponds to chlorophyll quantification results normalized by fresh weight. Error bars correspond to standard deviation between samples.

FIG. 6—A. Primary amino acid sequence alignment of BioF homologues (E. coli K12, B. subtilis 168 and B. paralicheniformis DSM31110, as indicated). Positions labelled with asterisks correspond to residues were identified SNPs result in an amino acid change. Squared-labelled positions correspond to catalytic residues. B. 3D structural model of BioFDSM31110 protein. Red-circle labels the position of the catalytic residues. Green boxes correspond to position of amino acid changes identified.

FIG. 7—Scheme representing the HrcA regulon described in B. subtilis. HrcA acts as transcriptional repressor of two operons, the heptacistronic dnak operon (hrcA-grpE-dnaK-dnaJ-3×orfs), and the bicistronic groE (groES-groEL). Analysis or the homologous genomic regions present in DSM31110 confirm conservation of these operons' organization.

FIG. 8. Plant growth promotion by cold adapted derivatives compared in A. thaliana grown in potting soil. Plant growth and fitness promotion quantification results from parental strain DSM33110 and 2 cold adapted improved derivative strains (CA4 and CA6 corresponding to CHCC36494-CHCC36496, respectively). A-B. Plant growth and fitness promotion quantification results from parental strain (DSM33110) and two combinations of cold adapted derivatives CA4 and CA6 with B. subtilis strains DSM33015 and DSM32938, respectively. A. thaliana seedlings pre-grown in potting soil for 7 days were inoculated by root dipping on bacterial cultures resuspended in 10 mM MgSO₄ buffer (OD₆₀₀ 0.01). Plantlets were allowed to grow for 14 additional days before data collection. Error bars correspond to standard deviation between replicates and statistical significance was determined by performing a t-test (p<0.05) assuming equal variance in the two samples. A. Plant growth experiments were grown in plant growth chamber at 22° C. B. Plant growth experiments were grown in plant growth chamber at 15° C.

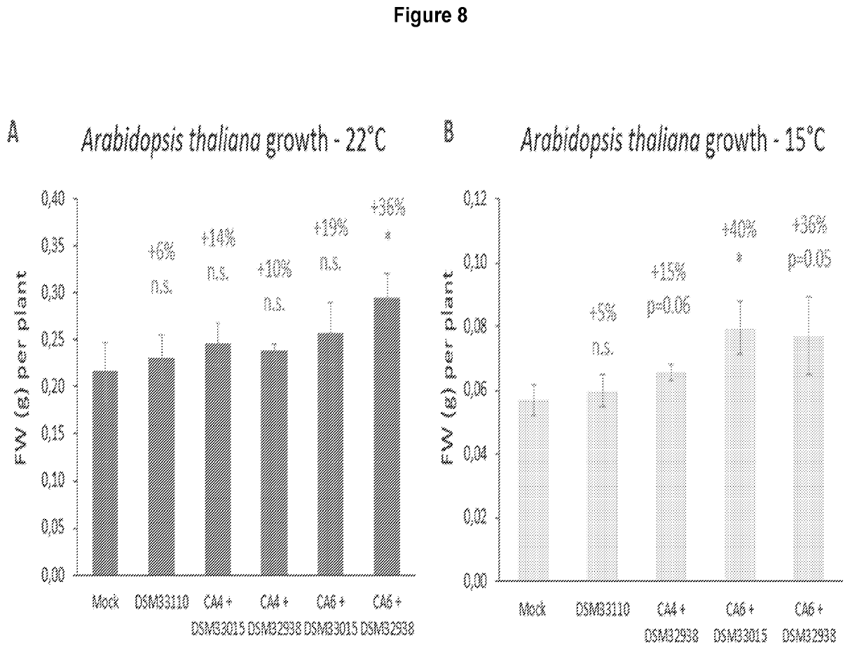

### DETAILED DESCRIPTION OF THE INVENTION

### Rhizobacteria

The soils are home to a complex biological community, of which micro-organisms, prokaryotes and eukaryotes form a majority, both in number and in diversity. Some prokaryotes have ecological niches as the rhizosphere, and/or the rhizoplane of plants, where they multiply, survive and protect themselves from the rest of the antagonistic action of soil microflora. These organisms have been generically called rhizobacteria.

In association with plants, rhizobacteria may have a deleterious effect, no effect or a beneficial effect. Those who exercise a beneficial effect—growth promotion and biological control of disease—are called PGPR (“Plant Growth-Promoting Rhizobacteria). It is estimated that only 1% to 2% of rhizobacteria have some beneficial effect for the plant with which they are associated.

### PGPR as Biocontrol Agents

PGPR have been used for biological control of plant diseases and thereby increase the productivity of crops. How and why this biological control is exercised, is still a topic that needs complementary studies.

In some situations, it is possible that biological control occurs by direct antagonism exerted by PGPR against the pathogen, with involvement of the known mechanisms of antibiosis: production of antimicrobial substances, direct parasitism, competition for nutrients and ecological niches.

Research has shown that certain PGPR appear to act as elicitor of ISR (induced systemic resistance), in the sense that the plant becomes systemically protected against more than one pathogen, unlike the classical biological control, which aims to implement the control more specifically.

A significant parameter affecting the PGPR ability to infect and colonize the plant surface is the ability of the PGPR to grow under the conditions to which it is exposed. The inventors of present disclosure therefore seek to improve the growth rates and biomass formation at different, and most preferably decreased temperatures and thereby improve the plant growth promoting capabilities the Bacilli disclosed herein.

### The Rhizosphere Environment

One of the most convenient methods of introducing a rhizobacteria in the root environment is through the application on the seeds before sowing. The process of seed germination releases carbohydrates and amino acids in abundance in the form of seed exudates. Thus, these organisms introduced with the seeds in the soil utilize exudates as a source of nutrition and colonize the roots as they emerge. Rhizobacteria isolates that have greater ability to utilize root exudates of seeds at different temperatures may have selective advantage in colonization of the roots.

PGPR of the genus Bacillus have been associated with nematode control. Sikora, R. A. (Interrelationship between plant health promoting rhizobacteria, plant parasitic nematodes and soil microorganisms. Medicine Faculty Landbouww Rijksuniv Gent, Landbouww, v.53, n.2b, p. 867-878, 1988) observed reductions in infection of Meloidogyne arenaria, M. incognita and Rotylenchulus reniformis around 60-65% with treatment of seeds of various crops with a strain of Bacillus.

### Advantages of Rhizobacteria for Commercial Application

The rhizobacteria have several advantages over chemical pesticides or even on other biological control agents: they are easy to mass-produce, they are easy to store and are adaptable to the formulation technology.

The rhizobacteria can be applied by treating the substrate, immersing the seedling root systems in bacterial suspensions, watering the plant with bacterial suspension by dipping/coating the seeds in suspension of rhizobacteria or by applying PGPR with the pelleting of seeds.

### Bacteria of the Genus Bacillus

The Bacillus species are Gram-positive bacteria characterized by having thick cell walls and the absence of outer membranes, which differs from the Gram-negative bacteria. Much of the cell wall of Gram-positive bacteria is composed of peptidoglycan.

Gram-positive species are divided into groups according to their morphological and biochemical characteristics. The genus Bacillus is belonging to the group of sporulating bacteria. Bacterial spores are one of the most resilient cell types; they resist many environmental changes, withstand dry heat and certain chemical disinfectants and may persist for years on dry land.

The beneficial effect of Bacilli such as e.g. B. paralicheniformis, when applied near the seed or the soil, may not be solely due to the antagonism afforded to pathogens. The PGPR has a positive influence on germination, development and crop yield due also to the production of substances which promote growth and improvement in plant nutrition by solubilization of phosphorus.

In the context of the present invention, a biofertilizer is a mixture of living microorganisms that when applied to seeds, plants or soil, promote the increase of nutrient supply, such as NH₄⁺, SO₄²⁻, K⁺ or PO₄³⁻ (Vessey, 2003).

In the context of the present invention, a plant biostimulant is any substance or microorganism applied to plants with the aim to enhance nutrition efficiency, abiotic stress tolerance and/or crop quality traits, regardless of its nutrients content. By extension, plant biostimulants also designate commercial products containing mixtures of such substances and/or microorganisms (du Jardin, 2015).

In the context of the present invention, plant growth promoting agent or plant growth promoting microorganism is a microorganism with the ability to colonize roots and/or inner plant tissues and promote plant growth and health by either acting as a biofertilizer, biostimulant or via biological control of plant disease. Said agent or microorganism is a soil and rhizosphere-inhabiting microorganism that can colonize plant roots in significant numbers (10⁵-10⁷ CFU per gram of fresh root) and influence plant growth in a positive manner (Spaepen et al. 2009; Antoun and Prevost 2005).

Thus, the first aspect of the invention relates to the herein described novel strains or mutants thereof.

The composition of the present invention may, besides the active components, contain agrochemical acceptable excipients and/or vehicles thereof. The composition of the invention further comprises agrochemically acceptable carriers, vehicles and/or adjuvants.

Among the main crops of plants are sugar cane, coffee, soybeans, cotton, corn, potatoes, tomatoes, tobacco, banana, rice, wheat, avocado, pineapple, squash, cacao, coconut, oats, onion, lettuce, beet, carrot, cassava, beans, sunflower, pepper, turnip, apple, strawberry, okra, radish and onion.

For fruticulture: citrus, grape, guava, papaya, fig, peach, plum and nespereira are of particular relevance and with regard to horticulture: eggplant and cruciferous.

For floriculture: rose, chrysanthemum, lisianthus, gerbera, amaryllis, begonia and celosia.

The composition of present invention may be coated on the plant seed and can include an amount of Bacillus, such as e.g. B. paralicheniformis spores from about 1.0×10² CFU/seed to about 1.0×10⁹ CFU/seed.

The plant seed can include, but is not limited to, the seed of monocots, dicots, Cereals, Corn, Sweet Corn, Popcorn, Seed Corn, Silage Corn, Field Corn, Rice, Wheat, Barley, Sorghum, Brassica Vegetables, Broccoli, Cabbage, Cauliflower, Brussels Sprouts, Collards, Kale, Mustard Greens, Kohlrabi, Bulb Vegetables, Onion, Garlic, Shallots, Fruiting Vegetables, Pepper, Tomato, Eggplant, Ground Cherry, Tomatillo, Okra, Grape, Herbs/Spices, Cucurbit Vegetables, Cucumber, Cantaloupe, Melon, Muskmelon, Squash, Watermelon, Pumpkin, Eggplant, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Lima beans, Peas, Chick peas, Split peas, Lentils, Oil Seed Crops, Canola, Castor, Cotton, Flax, Peanut, Rapeseed, Safflower, Sesame, Sunflower, Soybean, Root/Tuber and Corm Vegetables, Carrot, Potato, Sweet Potato, Beets, Ginger, Horseradish, Radish, Ginseng, Turnip, sugarcane, sugarbeet, Grass, or Turf grass.

Further, the plant seed can include seed of a drybean, a corn, a wheat, a soybean, a canola, a rice, a cucumber, a pepper, a tomato, a squash, a cotton, a grass, and a turf grass.

In an alternative embodiment, the Bacillus or composition of present invention may be added to: soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

In one or more embodiments, the plant can include soybean, bean, snap bean, wheat, cotton, corn, pepper, tomato, potato, cassava, grape, strawberry, banana, peanut, squash, pumpkin, eggplant, and cucumber.

In the compositions and methods of the present invention, the pathogenic infection can be caused by a wide variety of plant pathogens including, for example, but not limited to, a plant fungal pathogen, a plant bacterial pathogen, a rust fungus, a Botrytis spp., a Botrytis cinerea, a Botrytis squamosa, an Erwinia spp., an Erwinia carotovora, an Erwinia amylovora, a Dickeya spp., a Dickeya dadantii, a Dickeya solani, an Agrobacterium spp., a Agrobacterium tumefaciens, a Xanthomonas spp., a Xanthomonas axonopodis, a Xanthomonas campestris pv. carotae, a Xanthomonas pruni, a Xanthomonas arboricola, a Xanthomonas oryzae pv. oryzae, a Xylella spp., a Xylella fastidiosa, a Candidatus spp., a Candidatus liberibacter, a Fusarium spp., a Fusarium culmorum, a Fusarium graminearum, a Fusarium oxysporum, a Fusarium oxysporum f. sp. Cubense, a Fusarium oxysporum f. sp. Lycopersici, a Fusarium virguliforme, a Sclerotinia spp., a Sclerotinia sclerotiorum, a Sclerotinia minor, Sclerotinia homeocarpa, a Cercospora/Cercosporidium spp., an Uncinula spp., an Uncinula necator (Powdery Mildew), a Podosphaera spp. (Powdery Mildew), a Podosphaera leucotricha, a Podosphaera clandestine, a Phomopsis spp., a Phomopsis viticola, an Alternaria spp., an Alternaria tenuissima, an Alternaria porri, an Alternaria alternate, an Alternaria solani, an Alternaria tenuis, a Pseudomonas spp., a Pseudomonas syringae pv. Tomato, a Phytophthora spp., a Phytophthora infestans, a Phytophthora parasitica, a Phytophthora sojae, a Phytophthora capsici, a Phytophthora cinnamon, a Phytophthora fragariae, a Phytophthora spp., a Phytophthora ramorum, a Phytophthora palmivara, a Phytophthora nicotianae, a Phakopsora spp., a Phakopsora pachyrhizi, a Phakopsora meibomiae an Aspergillus spp., an Aspergillus flavus, an Aspergillus niger, a Uromyces spp., a Uromyces appendiculatus, a Cladosporium spp., a Cladosporium herbarum, a Rhizopus spp., a Rhizopus arrhizus, a Penicillium spp., a Rhizoctonia spp., a Rhizoctonia solani, a Rhizoctonia zeae, a Rhizoctonia oryzae, a Rhizoctonia caritae, a Rhizoctonia cerealis, a Rhizoctonia crocorum, a Rhizoctonia fragariae, a Rhizoctonia ramicola, a Rhizoctonia rubi, a Rhizoctonia leguminicola, a Macrophomina phaseolina, a Magnaorthe oryzae, a Mycosphaerella spp., Mycosphaerella graminocola, a Mycosphaerella fijiensis (Black sigatoga), a Mycosphaerella pomi, a Mycosphaerella citri, a Magnaporthe spp., a Magnaporthe grisea, a Monilinia spp., a Monilinia fruticola, a Monilinia vacciniicorymbosi, a Monilinia laxa, a Colletotrichum spp., a Colletotrichum gloeosporiodes, a Colletotrichum acutatum, a Colletotrichum Candidum, a Diaporthe spp., a Diaporthe citri, a Corynespora spp., a Corynespora Cassiicola, a Gymnosporangium spp., a Gymnosporangium juniperi-virginianae, a Schizothyrium spp., a Schizothyrium pomi, a Gloeodes spp., a Gloeodes pomigena, a Botryosphaeria spp., a Botryosphaeria dothidea, a Neofabraea spp., a Wilsonomyces spp., a Wilsonomyces carpophilus, a Sphaerotheca spp., a Sphaerotheca macularis, a Sphaerotheca pannosa, a Erysiphe spp., a Stagonospora spp., a Stagonospora nodorum, a Pythium spp., a Pythium ultimum, a Pythium aphanidermatum, a Pythium irregularum, a Pythium ulosum, a Pythium lutriarium, a Pythium sylvatium, a Venturia spp, a Venturia inaequalis, a Verticillium spp., a Ustilago spp., a Ustilago nuda, a Ustilago maydis, a Ustilago scitaminea, a Claviceps spp., a Claviceps puprrea, a Tilletia spp., a Tilletia tritici, a Tilletia laevis, a Tilletia horrid, a Tilletia controversa, a Phoma spp., a Phoma glycinicola, a Phoma exigua, a Phoma lingam, a Cocliobolus sativus, a Gaeumanomyces gaminis, a Colleototricum spp., a Rhychosporium spp., Rhychosporium secalis, a Biopolaris spp., a Helminthosporium spp., a Helminthosporium secalis, a Helminthosporium maydis, a Helminthosporium solai, and a Helminthosporium tritici-repentis, or combinations thereof.

In some embodiments, the pathogenic infection can be caused by one or a combination of: Soybean rust fungi (Phakopsora pachyrhizi, Phakopsora meibomiae) and the plant comprises soybean; Botrytis cinerea (Botrytis Blight) and the plant comprises grape; Botrytis cinerea (Botrytis Blight) and the plant comprises strawberry; Botrytis cinerea (Botrytis Blight) and the plant comprises tomato; Alternaria spp. (e.g. A. solani) and the plant comprises tomato; Alternaria spp. (e.g. A. solani) and the plant comprises potato; Bean Rust (Uromyces appendiculatus) and the plant comprises common bean; Microsphaera diffusa (Soybean Powdery Mildew) and the plant comprises soybean; Mycosphaerella fijiensis (Black sigatoga) or Fusarium oxysporum f. sp. cubense (Panama disease) and the plant comprises banana; Xanthomonas spp, or Xanthomonas oryzae pv. oryzae and the plant comprises rice; Xanthomonas axonopodis and the plant comprises cassava; Xanthomonas campestris and the plant comprises tomato; Botrytis cinerea (Pepper Botrytis Blight) and the plant comprises pepper; Powdery mildew and the plant comprises a cucurbit; Sclerotinia sclerotiorum (white mold) and the plant comprises snap bean; Sclerotinia sclerotiorum (white mold) and the plant comprises potato; Sclerotinia homeocarpa (dollar spot) and the plant comprises turfgrass; Southern White Mold and the plant comprises peanut; Leaf spot (Cercospora/Cercosporidium) and the plant comprises peanut; Fusarium graminearum (Wheat Head Scab) and the plant comprises wheat; Mycosphaerella graminicola (Septoria tritici blotch) and the plant comprises wheat; Stagonospora nodorum (glume blotch and Septoria nodorum blotch), and the plant compromises wheat; Erwinia amylovora, and the plant compromises apple, pear and other pome fruits; Venturia inaequalis, and the plant compromises apple, pear and other pome fruits; or Rhizoctonia solani and the plant comprises wheat, rice, turfgrass, soybean, corn, legumes and vegetable crops. The compositions including the bacilli as described herein strain can be in the form of a liquid, an oil dispersion, a dust, a dry wettable powder, a spreadable granule, or a dry wettable granule. More specifically the composition may for example be an emulsion concentrate (EC), a suspension concentrate (SC), a suspo-emulsion (SE), a capsule suspension (CS), a water dispersible granule (WG), an emulsifiable granule (EG), a water in oil emulsion (EO), an oil in water emulsion (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a dispersible concentrate (DC), a wettable powder (WP) or any technically feasible formulation in combination with agriculturally acceptable adjuvants.

Hence, the present invention relates to a composition comprising Bacillus paralicheniformis DSM 33238, DSM 33239, DSM 33240, DSM 33241, DSM 33242, DSM 33243, DSM 33244 or mutants thereof or a mutant thereof, and to a kit comprising the composition, or prepared by the process of preparing the composition, as well as instructions and a suitable recipient. Accordingly, the present invention also relates to a process for preparing a composition comprising Bacillus paralicheniformis DSM 33238, DSM 33239, DSM 33240, DSM 33241, DSM 33242, DSM 33243, DSM 33244, or a mutant thereof together with agrochemically acceptable carriers, vehicles and/or adjuvants, and use of said composition for controlling, combating and/or conferring specific resistance to phytonematodes are also given.

In addition, the invention refers to the use of effective amounts of Bacillus paralicheniformis DSM 33238, DSM 33239, DSM 33240, DSM 33241, DSM 33242, DSM 33243, DSM 33244 or a mutant thereof, in the manufacture of an agrochemical composition with plant growth promoting effect in a plant culture, as well as processes for promoting plant health.

In a preferred aspect the invention relates to a Bacillus having a mutation in the bioF gene, wherein the mutation changes the enzyme kinetics of the protein encoded by bioF, when compared to the parental strain of the Bacillus having a mutation in the bioF gene.

In yet a preferred aspect the invention relates to a Bacillus having a mutation in the hrcA gene, wherein the mutation renders the protein encoded by hrcA dysfunctional such loss of function, when compared to its parental strain.

Further, the present invention relates to the following aspects:
Aspect 1. A Bacillus having mutation in the bioF and/or hrcA gene.
Aspect 2. A Bacillus having a mutation in the hrcA and/or bioF gene when compared to the corresponding ortholog genes of hrcA and bioF in B. paralicheniformis deposited as DSM31110.
Aspect 3. A Bacillus having a mutation in the hrcA gene when compared to SEQ ID NO: 1 and/or a mutation in the bioF gene when compared to the SEQ ID NO: 3.
Aspect 4. A Bacillus selected from a list consisting of the strains deposited at Deutsche Sammlung von Mikroorganismen und Zellkulturen with accession No's. DSM 33238, DSM 33239, DSM 33240, DSM 33241, DSM 33242, DSM 33243, DSM 33244.
Aspect 5. A Bacillus according to any of the preceding aspects, wherein the closest ortholog of the bioF gene of said Bacillus share less than 100% such as e.g. less than 99%, less than 98% sequence identity with SEQ ID NO: 1.
Aspect 6. A Bacillus according to any of the preceding aspects, wherein the closest ortholog of the bioF gene of said Bacillus share at least 95% such as e.g. at least 96%, at least 97, at least 98, at least 99% sequence identity with SEQ ID NO:1.
Aspect 7. A Bacillus according to any of the preceding aspects, wherein the closest ortholog of the hrcA gene of said B. paralicheniformis share less than 100% such as e.g. less than 99%, less than 98% sequence identity with SEQ ID NO:3.
Aspect 8. A Bacillus according to any of the preceding aspects, wherein the closest ortholog of the hrcA gene of said Bacillus share at least 95% such as e.g. at least 96%, at least 97 sequence identity with SEQ ID NO: 3.
Aspect 9. A Bacillus according to any of the preceding aspects, wherein the mutation is a deletion, substitution or insertion.
Aspect 10. A Bacillus according to any of the preceding aspects, wherein the mutation causes a frameshift, introduces a stop codon or impacts the kinetics of the encoded protein.
Aspect 11. A Bacillus according to any of the preceding aspects, wherein the protein encoded by one or more of the genes hrcA and/or bioF or one or more of their closest orthologs is structurally impacted by the mutation.
Aspect 12. A Bacillus according to any of the preceding aspects, wherein the genome of the strain is at least 99%, such as e.g. at least 99.5%, such as e.g. at least 99.8%, such as e.g. at least 99.9% identical to the genome of the strain deposited at Deutsche Sammlung von Mikroorganismen und Zellkulturen with accession No. DSM31110.
Aspect 13. A Bacillus according to any of the preceding aspects, wherein the Bacillus is selected from: Bacillus licheniformis, Bacillus amyloliquefaciens, Bacillus paralicheniformis, Bacillus cereus, Bacillus velezensis, Bacillus megaterium.
Aspect 14. A Bacillus according to any of the preceding aspects, wherein the strain is derived from the strain deposited at Deutsche Sammlung von Mikroorganismen und Zellkulturen with accession No. DSM31110 or a strain sharing pheno- or genotypical characteristics with the strain deposited at Deutsche Sammlung von Mikroorganismen und Zellkulturen with accession No. DSM31110
Aspect 15. A Bacillus according to any of the preceding aspects wherein the Bacillus has the pheno- or genotypical characteristics of one or more of the Bacillus paralicheniformis strains deposited at Deutsche Sammlung von Mikroorganismen und Zellkulturen with accession No's. DSM 33238, DSM 33239, DSM 33240, DSM 33241, DSM 33242, DSM 33243, DSM 33244.
Aspect 16. A Bacillus according to any of the preceding aspects showing increased growth rates when compared with the Bacillus paralicheniformis deposited at Deutsche Sammlung von Mikroorganismen und Zellkulturen with accession no. DSM31110, when grown at 21° C. in MSgg and/or MS-rex medium.
Aspect 17. A Bacillus according to aspect 16 wherein the growth rate is determined as described in Example 2 herein.
Aspect 18. A Bacillus according to any of aspects 16 or 17 where the growth rate exceeds the growth rate of the parental strain by at least 20%, such as at least 40%, such as at least 60%, such as at least 80%, such as at least 100%.
Aspect 19. A composition comprising a Bacillus according to any of the preceding aspects.
Aspect 20. A composition comprising a Bacillus according to any of the preceding aspects and agrochemically acceptable excipients and/or carriers thereof.
Aspect 21. The composition of any of aspects 19 or 20, further comprising one or a combination of a microbial, a biological, or a chemical insecticide, fungicide, nematicide, bactericide, herbicide, plant extract, plant growth regulator, or fertilizer present in an amount suitable to benefit plant growth and/or to confer protection against a pathogenic infection in a susceptible plant, a carrier, a surfactant, a dispersant, or a yeast extract.
Aspect 22. Use of a composition according to any of aspects 19 to 21 or a Bacillus according to any of aspects 1 to 18 as a biostimulant and/or bionematicide.
Aspect 23. Use of a composition, according to any of aspects 19 to 21, or a Bacillus according to any of aspects 1 to 18 for controlling, combating and/or conferring specific resistance to phytonematodes.
Aspect 24. Use according to any of aspects 22 or 23, wherein the phytonematodes are selected from the group consisting of Meloidogyne, Pratylenchus, Heterodera, Globodera, Ditylenchus, Tylenchulus, Xiphinema, Radopholus, Rotylenchulus, Helicotylenchus and Belonolaimus.
Aspect 25. Use according to any of aspects 22 to 24, wherein the phytonematode is selected from the group consisting of Meloidogyne incognita, Meloidogyne javanica, Meloidogyne exigua, Meloidogyne paranaensis, Heterordera glycines and Pratylenchus zeae.
Aspect 26. Use according to any of aspects 22 to 25 wherein the composition according to any of aspects 19 to 21 or the Bacillus according to any of aspects 1 to 18 is applied on a plant, a seed or in the habitat of a plant
Aspect 27. Use according to aspect 26 wherein the plant is selected from the group consisting of corn, rice, sugar cane, soybean, potato, carrot, coffee and banana.
Aspect 28. Process for conferring improved resistance to phytonematodes, comprising applying an effective amount of a Bacillus of any of aspects 1 to 18 or a composition according to any of aspects 19 to 21 on plants and/or their habitat.
Aspect 29. Kit, comprising the composition as defined in any one of aspects 19 to 21, instructions for use and a suitable container.
Aspect 30. A plant seed coated with a composition according to any of aspects 19 to 21 present in an amount suitable to benefit plant growth and/or to confer protection against a pathogenic infection in a susceptible plant.
Aspect 31. The plant seed of aspect 30, wherein the composition comprises an amount of spores of the Bacillus of any of aspects 1 to 18 from about 1.0×10² CFU/seed to about 1.0×10⁹ CFU/seed.
Aspect 32. The plant seed of aspect 30 or 312, wherein the composition further comprises one or a combination of a microbial, a biological, or a chemical insecticide, fungicide, nematicide, bactericide, or plant growth regulator present in an amount suitable to benefit plant growth and/or to confer protection against a pathogenic infection in a susceptible plant.
Aspect 33. A Bacillus according to any of aspects 1 to 18 wherein BioF is encoded by the DNA sequence of SEQ ID NO: 1 or amino acid sequence of SEQ ID NO: 2 or homologs thereof and/or HrcA is encoded by the DNA sequence of SEQ ID NO: 3 or amino acid sequence homologs thereof.
Item 1. A Bacillus paralicheniformis having a mutation in the hrcA and/or bioF gene when compared to the corresponding ortholog genes of hrcA and/or bioF in B. paralicheniformis deposited at Deutsche Sammlung von Mikroorganismen und Zellkulturen (DSMZ) with accession no. DSM31110.
Item 2. A Bacillus paralicheniformis having a mutation in the hrcA and/or bioF gene when compared to the corresponding ortholog genes of hrcA encoded by SEQ ID NO: 1 and/or bioF encoded by SEQ ID NO: 3 in e.g. B. paralicheniformis deposited at Deutsche Sammlung von Mikroorganismen und Zellkulturen (DSMZ) as DSM31110.
Item 3. A Bacillus paralicheniformis according to any of items 1 or 2, wherein the Bacillus paralicheniformis is a mutant derived from B. paralicheniformis deposited as DSM31110.
Item 4. A Bacillus paralicheniformis deposited at Leibniz Institute DSMZ—German Collection of Microorganisms and Cell Cultures, Inhoffenstr. 7B, D-38124 Braunschweig (DSMZ) as DSM 33238, DSM 33239, DSM 33240, DSM 33241, DSM 33242, DSM 33243 or DSM 33244.
Item 5. A Bacillus paralicheniformis according to any of items 1 to 4 wherein the mutation in the bioF gene causes a genotypic change imposing an amino acid change from Pro314 to Ser, Ala244 to Val, Asp300 to Gly and/or Arg37 to Trp.
Item 6. A Bacillus paralicheniformis according to any of items 1 to 5 wherein the mutation in the hrcA gene causes a genotypic change imposing a stop codon, rendering an inactive HrcA repressor.
Item 7. A Bacillus paralicheniformis according to any of the preceding items showing increased maximum growth rates when compared with the Bacillus paralicheniformis deposited at Deutsche Sammlung von Mikroorganismen und Zellkulturen (DSMZ) with accession no. DSM31110, when grown at 21° C. in MSgg.
Item 8. A Bacillus paralicheniformis according to item 7 wherein the maximum growth rate is determined as described in Example 2 herein.
Item 9. A Bacillus paralicheniformis according to any of the preceding items wherein the growth rate of the Bacillus paralicheniformis strain according to any of the preceding items exceeds the growth rate of the Bacillus paralicheniformis deposited at Deutsche Sammlung von Mikroorganismen und Zellkulturen (DSMZ) under accession no. DSM31110 by at least 20%, such as at least 40%, such as at least 60%, such as at least 80%, such as at least 100%.
Item 10. A Bacillus paralicheniformis according to any of the preceding claims which when applied to a Arabidopsis thaliana Col-0 plants, in a gnotobiotic plant system and grown for 22° C. and/or 15° C. is able to increase the total leaf area, the fresh weight or the chlorophyll content in said A. thaliana.
Item 11. A composition comprising a Bacillus paralicheniformis according to any of the preceding items and agrochemically acceptable excipients and/or carriers thereof.
Item 12. A composition of item 11, further comprising one, or a combination of a microbial, a biological, or a chemical insecticide, fungicide, nematicide, bactericide, herbicide, plant extract, plant growth regulator, or fertilizer present in an amount suitable to benefit plant growth and/or to confer protection against a pathogenic infection in a susceptible plant, a carrier, a surfactant, a dispersant, or a yeast extract.
Item 13. Use of a composition according to any of items 11 to 12 or a Bacillus paralicheniformis according to any of items 1 to 9 as a biostimulant, growth promoter and/or bionematicide.
Item 14. Use according to item 13, wherein the phytonematodes are selected from the group consisting of Meloidogyne, Pratylenchus, Heterodera, Globodera, Ditylenchus, Tylenchulus, Xiphinema, Radopholus, Rotylenchulus, Helicotylenchus and Belonolaimus such as e.g. Meloidogyne incognita, Meloidogyne javanica, Meloidogyne exigua, Meloidogyne paranaensis, Heterordera glycines and Pratylenchus zeae.
Item 15. Use according to any of items 13 to 14 wherein the composition according to any of items 11 to 12 or the Bacillus licheniformis according to any of items 1 to 10 is applied on a plant, a seed or in the habitat of a plant, such as in the soil.
Item 16. Use according to any of items 13 to 15 wherein the plant is selected from the group consisting of corn, rice, sugar cane, soybean, potato, carrot, coffee and banana.
Item 17. A plant seed coated with a composition according to any of items 11 to 12 present in an amount suitable to benefit plant growth and/or to confer protection against a pathogenic infection in a susceptible plant.
Item 18. The plant seed of item 17, wherein the composition comprises an amount of spores of the Bacillus of any of items 1 to 10 from about 1.0×10² CFU/seed to about 1.0×10⁹ CFU/seed.
Item 19. The plant seed of item 18 or 18, wherein the composition further comprises one or a combination of a microbial, a biological, or a chemical insecticide, fungicide, nematicide, bactericide, or plant growth regulator present in an amount suitable to benefit plant growth and/or to confer protection against a pathogenic infection in a susceptible plant.

The illustrative examples presented below serve to better describe the present invention. However, the formulations described merely refer to some means to some embodiments of the present invention and should not be taken as limiting the scope thereof.

As used in present disclosure the strain descriptors are used interchangeably according to the table below:

| | |
|---|---|
| CHCC32528 | CA1 |
| CHCC32529 | CA2 |
| CHCC32530 | CA3 |
| CHCC36494 | CA4 |
| CHCC36495 | CA5 |
| CHCC36496 | CA6 |
| CHCC36497 | CA7 |
| CHCC36751 | CAREX1 |
| CHCC36752 | CAREX2 |
| CHCC36753 | CAREX3 |
| CHCC36754 | CAREX4 |
| CHCC36755 | CAREX5 |
| CHCC36756 | CAREX6 |
| CHCC36757 | CAREX7 |

### EXAMPLES

### Growth Media Compositions:

### MSgg Medium (pH 7) (Filter Sterilized):

### MOPS Solution 1×:

100 mM morpholinepropanesulfonic acid (MOPS) (pH 7)
5 mM KH2PO4/K2HPO4

### 1× Trace Elements Solution:

50 μM MnCl2
1 μM ZnCl2
100 μM FeCl3
2 mM MgCl2
700 μM CaCl2
2 μM thiamine

### Aminoacid Supplements

50 μg/ml threonine, tryptophan and/or phenylalanine

### Carbon Sources

0.5% glutamate
0.5% glycerol

### MS-REX (pH 7) Filter Sterilized

Murashige and Skoog (MS) medium is a plant growth medium used in the laboratories for cultivation of plants or plant cell cultures. MS was supplemented with corn root exudates (1×) collected from hydroponic maize plants cultures. Root exudates had been collected in water, and liophylized to obtain a concentrate of the root exudates (×25).

### MS Composition

### Major Salts (Macronutrients)/1 L

Ammonium nitrate (NH4NO3) 1,650 mg/l
Calcium chloride (CaCl2·2H2O) 440 mg/l
Magnesium sulfate (MgSO4·7H2O) 370 mg/l
Monopotassium phosphate (KH2PO4) 170 mg/l
Potassium nitrate (KNO3) 1,900 mg/l.

### Minor Salts (Micronutrients)/1 L

Boric acid (H3BO3)6·2 mg/l
Cobalt chloride (CoCl2·6H2O) 0.025 mg/l
Ferrous sulfate (FeSO4·7H2O) 27.8 mg/l
Manganese (II) sulfate (MnSO4·4H2O) 22.3 mg/l
Potassium iodide (KI) 0.83 mg/l
Sodium molybdate (Na2MoO4·2H2O) 0.25 mg/l
Zinc sulfate (ZnSO4·7H2O) 8.6 mg/l
Ethylenediaminetetraacetic acid ferric sodium (NaFe-EDTA) constituting 5 ml/l of a stock solution containing 5.57 g FeSO4·7H₂O and 7.45 g Na2-EDTA per litre of water.
Copper sulfate (CuSO4·5H2O) 0.025 mg/l
Vitamins and organic compounds/1 L
Myo-Inositol 100 mg/l
Nicotinic Acid 0.5 mg/l
Pyridoxine·HCl 0.5 mg/l
Thiamine·HCl 1.0 mg/l
Glycine 2 mg/l

### Example 1: Adaptive Laboratory Evolution (ALE)

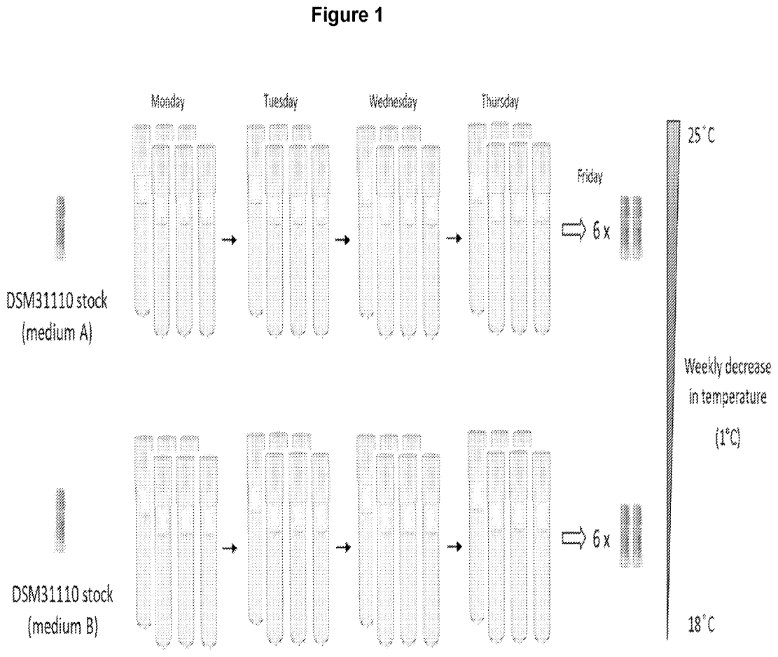

We performed an adaptive laboratory evolution (ALE) campaign to develop derivatives of B. paralicheniformis DSM31110 with improved growth rates at cold temperatures. Two different growth media were selected to carry on the evolution process in parallel. 12 independent cultures (6 per growth medium) were grown over-night and diluted every day to fresh medium (see FIG. 1). Initially, MSgg or MS-Rex growth media were inoculated from a stock of Bacillus paralicheniformis DSM31110 cell culture (OD600 1). Optical density of DSM31110 subcultures was monitored daily and subsequently diluted to fresh growth medium. Two glycerol stocks were prepared per tube weekly. One was used for inoculation of a new round of growth passages, while the second one was kept frozen. Temperature was gradually decreased from 25° C. to 18° C. throughout the 12 weeks of ALE campaign. Total number of generations reached was counted, and after 12 weeks of adaptive evolution, cultures were grown for 120-140 generations. Final samples from the evolution experiment were serially diluted and plated on LB agar plates to obtain isolated colonies. 2×960 clones were picked with a colony picker robot and grown in 96 well plates to generate individual glycerol stocks.

### Example 2: Growth Rate Analysis

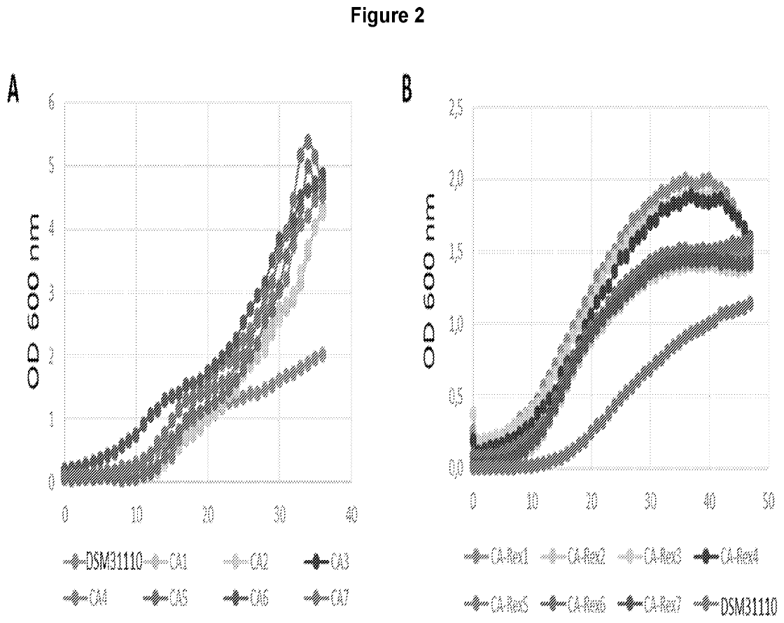

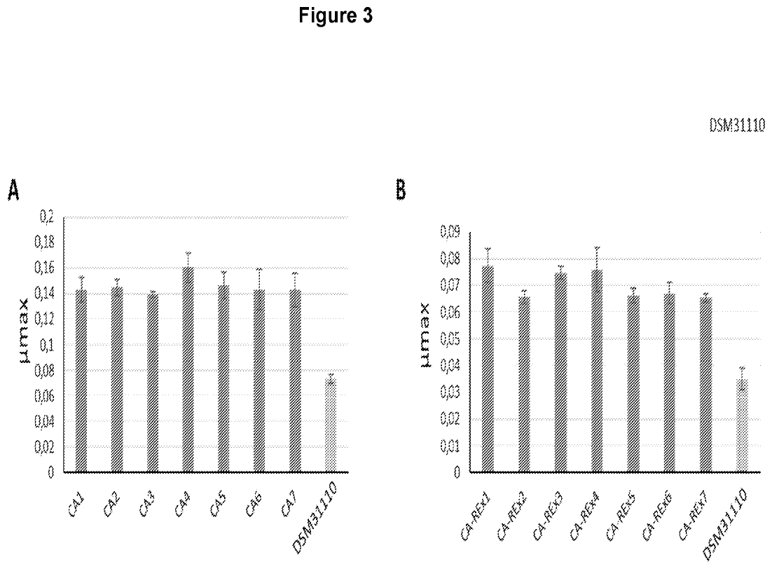

To compare growth profiles and growth rates between adapted derivatives and the parental strain, 2×958 clones (plus parental strain DSM31110) were inoculated into 96-deep-well plates containing either MSgg or MS-Rex medium. Growth experiments were performed in a Growth Profiler at 21° C. Data obtained allowed identification of the faster growing derivatives and selection of best 30 individual clones. Growth curves were then determined in triplicates to ensure reproducibility. Growth rates were calculated applying the slope function to OD600 recorded values in exponential phase. Umax values correspond to change in OD600 unit per hour (h⁻¹) FIG. 2 shows average growth data from the seven best derivatives developed from each growth media. FIG. 3 shows calculated maximum growth rates (μmax) for derivatives and mother strain DSM31110.

### Example 3—Genome Sequencing and SNP Analysis of Improved Derivatives

Final selected clones (2×7) were deposited in the CHCC collection and genome sequenced. Single nucleotide polymorphism (SNP) analysis was performed by comparing the genome sequences of the selected cold adapted strains to the DSM31110 genome sequence.

TABLE 1
SNP analysis and ORF target identification. Table contains
information linking cold adapted derivatives ID/name with the corresponding
CHCC number. SNPs identity and frequency with which they are found are
summarized.

| CHCC | Strain | | |
|---|---|---|---|
| number | name | SNPs analysis | Frequency |
| CHCC32528 | CA1 | 8-amino-7-oxononanoate synthase (EC2.3.1.47): | 8 out of 14 |
| | | p.Pro314Ser (BioF) | (57%) |
| | | YbgE homologue-Branched-chain amino acid | 2 out of 14 |
| | | aminotransferase (EC2.6.1.42): p.Asp3fs | (14%) |
| CHCC32529 | CA2 | 8-amino-7-oxononanoate synthase (EC2.3.1.47): | 8 out of 14 |
| | | p.Ala64Val (BioF) | (57%) |
| | | Flagellar biosynthesis protein FIiP: p.Lys156Glu | 1 out of 14 |
| | | Smc-superfamily protein: p.Ser163fs | 1 out of 14 |
| CHCC32530 | CA3 | Heat-inducible transcription repressor HrcA: p.Gln6* | 5 out of 14 |
| | | SNP in region upstream of BioWAFDB operon. | 2 out of 14 |
| | | Maybe affecting the promoter region | (10/14) |
| CHCC36494 | CA4 | 8-amino-7-oxononanoate synthase (EC2.3.1.47): | 8 out of 14 |
| | | p.Arg37Trp (BioF) | (57%) |
| | | Flagellar biosynthesis protein FIhA: p.Ala128Val | 3 out of 14 |
| | | | (21%) |
| CHCC36495 | CA5 | SNP in region upstream of BioWAFDB operon. | 2 out of 14 |
| | | Maybe affecting the promoter region | (9/14) |
| CHCC36496 | CA6 | 8-amino-7-oxononanoate synthase (EC 2.3.1.47): | 8 out of 14 |
| | | p.Pro314Ser (BioF) | (57%) |
| | | YbgE homologue-Branched-chain amino acid aminotransferase | 2 out of 14 |
| | | (EC 2.6.1.42): p.Phe195fs | (14%) |
| | | Flagellar biosynthesis protein FIhB: p.Glu16fs | 1 out of 14 |
| | | | (7/14) |
| CHCC36497 | CA7 | Heat-inducible transcription repressor HrcA: p.Pro314Thr | 5 out of 14 |
| | | ABC transporter permease protein: p.Lys624fs | 1 out of 14 |
| CHCC36751 | CAREX1 | Glycogen synthase, ADP-glucose transglucosylase (EC | 1 out of 14 |
| | | 2.4.1.21): p.Lys150fs | 5 out of 14 |
| | | Heat-inducible transcription repressor HrcA: p.Gln11* | |
| CHCC36752 | CAREX2 | 8-amino-7-oxononanoate synthase (EC 2.3.1.47): | 8 out of 14 |
| | | p.Ala244Val (BioF) | (57%) |
| | | Flagellar biosynthesis protein FIhA: p.Ala128Val | 3 out of 14 |
| | | | (21%) |
| CHCC36753 | CAREX3 | Heat-inducible transcription repressor HrcA: p.Gln11* | 5 out of 14 |
| CHCC36754 | CAREX4 | 8-amino-7-oxononanoate synthase (EC2.3.1.47): | 8 out of 14 |
| | | p.Pro314Ser (BioF) | (57%) |
| | | SNP in promoter region of YpfA homologue, receptor | 2 out of 14 |
| | | of c-di-GMP: T to C | 2 out of 14 |
| | | Flagellar basal-body rod protein FIgG: p.Gln152* | 1 out of 14 |
| | | Hypothetical protein in operon with YteA, | |
| | | a RsbR paralogue:: p.Phe5fs. | |
| CHCC36755 | CAREX5 | 8-amino-7-oxononanoate synthase (EC 2.3.1.47): | 8 out of 14 |
| | | p.Asp309Gly (BioF) | (57%) |
| | | Transcriptional regulator GabR of GABA utilization: p.Arg30Leu | 1 out of 14 |
| | | SNP in promoter region of YpfA homologue, receptor of | 2 out of 14 |
| | | c-di-GMP: T to C | 2 out of 14 |
| | | Flagellar basal-body rod protein FIgG: p.Gln152* | |
| CHCC36756 | CAREX6 | 8-amino-7-oxononanoate synthase (EC 2.3.1.47): p.Arg37Trp | 8 out of 14 |
| | | Flagellar biosynthesis protein FIhA: p.Ile182fs and several other | (57%) |
| | | SNPs | 3 out of 14 |
| | | | (21%) |
| CHCC36757 | CAREX7 | Heat-inducible transcription repressor HrcA: p.Gln11* | 5 out of 14 |

Out of 14 cold adapted DSM31110 derivatives sequenced, 8 showed mutations targeting the bioF orf. bioF encodes an 8-amino-7-oxononanoate synthase (EC2.3.1.47), enzyme in the biotin cofactor biosynthetic pathway. See below for physiological interpretation of the SNPs identified.

Genome sequencing results revealed the presence of several SNPs (found in 5 out of 14 derivatives) within the orf hrcA, encoding the heat-inducible transcriptional repressor HrcA. In most cases the identified mutations correspond to stop codons, and only in one of the mutant strains we identified an amino acid change Pro314 to Thr. Most likely all those mutations render an inactive repressor.

### Example 4—Growth Advantage Specificity Analysis

To assess whether the growth improvement phenotype was restricted to a specific growth medium composition or low temperatures, we compare the growth profiles of cold-adapted derivatives with the parental strain DSM31110 in complex media (LB and BHI) at 30° C. temperature.

Higher biomass yields and faster growth rates were observed for the cold adapted derivatives, suggesting that the mutations do not only help the strains to grow at low temperatures. During the adaptive evolution process we have selected for fast growers and derivatives that reach higher yields.

### Example 5—Plant Growth Promotion Experiments

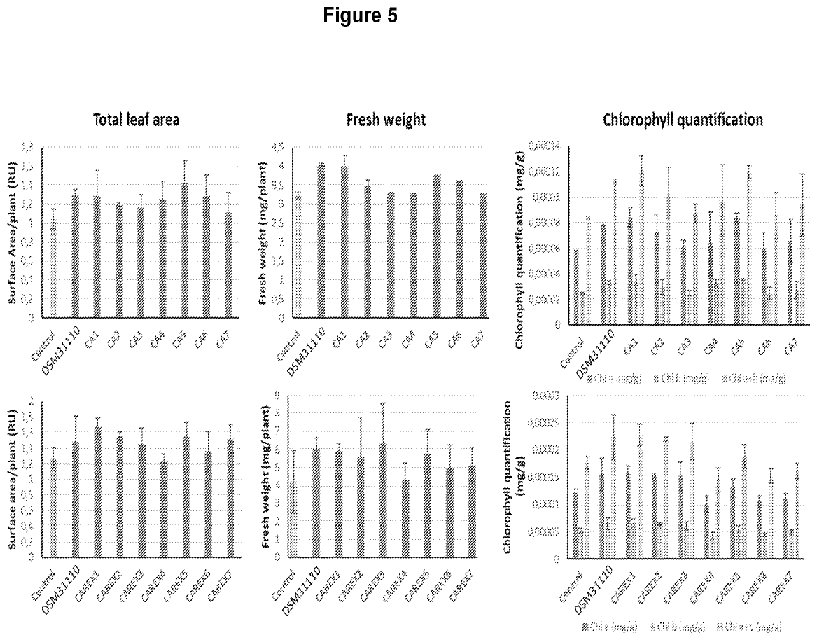

Plant growth promotion efficiency was compared between the derivative strains at 22° C. and 15° C. using A. thaliana Col-0 plants, in a gnotobiotic system based on the use of 24-well plates filled with plant growth medium solidified with agar. Based on the results from three independent experiments, both in soil and agar systems, derivative strains CA4, CA6 and CAREX1 were selected (CHCC36494, CHCC36496, and CHCC36751, respectively) as the best cold adapted DSM31110 derivatives. Results shown in FIG. 5.

To further evaluate the plant fitness promotion effect of cold adapted selected derivatives, a series of experiments were setup by the application team. Plant growth promotion efficiency was compared between derivative strains using A. thaliana plants in a soil system. Experiments were performed in plant growth chambers. Two different strain combinations containing cold adapted improved derivatives CA4 and CA6 were compared with the parental strain in their ability to promote A. thaliana plant growth. Strains B. paralicheniformis CHCC36494 (CA4) and CHCC36496 (CA6) were co-inoculated with 2 different Bacillus subtilis strains, as indicated in FIG. 19. A. thaliana seedlings were germinated and pre-grown in potting soil for 7 days prior inoculation. Bacterial cultures were grown over-night the day before inoculation. In the morning of inoculation day, bacterial cultures were diluted and grown to OD₆₀₀ 1 in LB broth. Bacterial cells were then washed and resuspended into 10 mM MgSO₄ buffer at OD₆₀₀ 0.01. Plants were inoculated with 1e6 CFUs (8 plant replicates per strain), comparing the results from control non-inoculated plants with the parental strain and cold adapted derivatives. Plants grew in growth chambers for 2 weeks, with 16 h light/8 h dark photoperiod at two different temperatures (22° C. and 15° C.) to evaluate the performance of these strains under lower, suboptimal Ta conditions. Shoot fresh weight quantification was done 2 weeks post-inoculation. Results obtained showed an increase in plant growth for all treatments compared to non-inoculated plants, with a more relevant increase being observed for the two combinations containing the cold adapted improved derivative CA6 (19%-40% and 36%-36% increase, at 22° C. and 15° C. temperature, respectively). Both at 22° C. and 15° C., strain combinations containing derivative CA6 (CHCC36496) returned the best results. Nevertheless, only one strain combination showed statistically significant results at 15° C. compared to non-inoculated plants. Therefore, strain combination CA6 (CHCC36496) plus B. subtilis DSM33015 was selected to carry on additional testing in crop plants and field trials.

### Example 6—Physiological Interpretation of MoA Associated with Identified SNPS

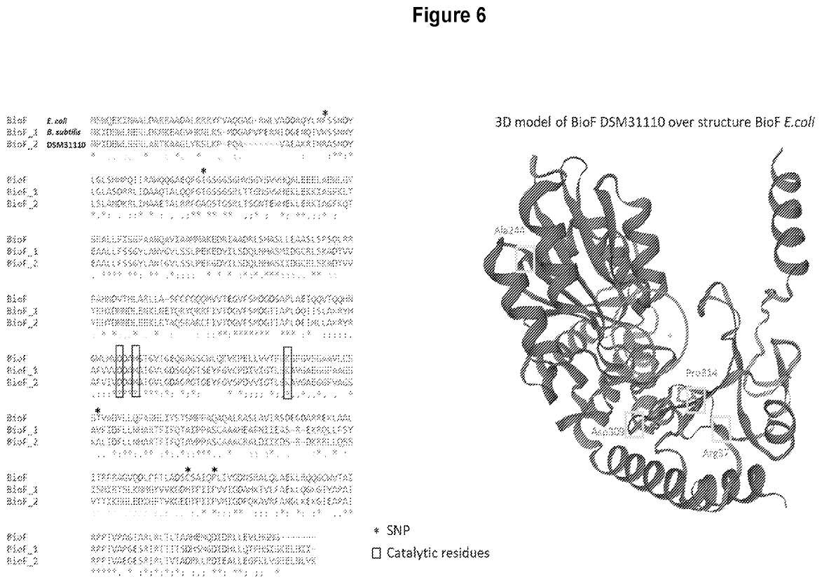

BioF-Biotin (also known as vitamin H) is a covalently bound enzyme cofactor required by all forms of life. In 5 out the 8 mutated sequences, the genotypic change renders a protein with an amino acid change from Pro 314 to Ser. In the other 3 mutated sequences the amino acid changes are different (Ala244 to Val, Asp300 to Gly and Arg37 to Trp). Homology searches, ClustalW alignments, and protein 3D structural modelling of the BioF DSM31110 amino acid sequence suggest that selected SNPs result in amino acid changes that most likely are not part of the catalytic site (1) (FIG. 6). How those SNPs affect the activity of the BioF enzyme and intracellular levels of biotin is currently under investigation.

Biotin is an essential cofactor required for diverse key metabolic enzymes that carry out carboxylation and decarboxylation reactions in fatty acid synthesis, amino acid metabolism and gluconeogenesis (2). In B. cereus, limiting amounts of biotin were reported to restrict growth and alter the cell membrane fatty acid composition (3). In Bacillus spp., the first intermediate in fatty acid and phospholipid synthesis is malonyl-CoA. Malonyl-CoA is synthesized from acetyl-CoA by the acetyl-CoA carboxylase, encoded on the accBC genes. The accBC operon from Bacillus codes for two subunits of acetyl-CoA carboxylase, biotin carboxyl-carrier and biotin carboxylase. In addition, a direct correlation between the levels of transcription of the fatty acid accBC genes and the rate of cellular growth was reported in B. subtilis (4)

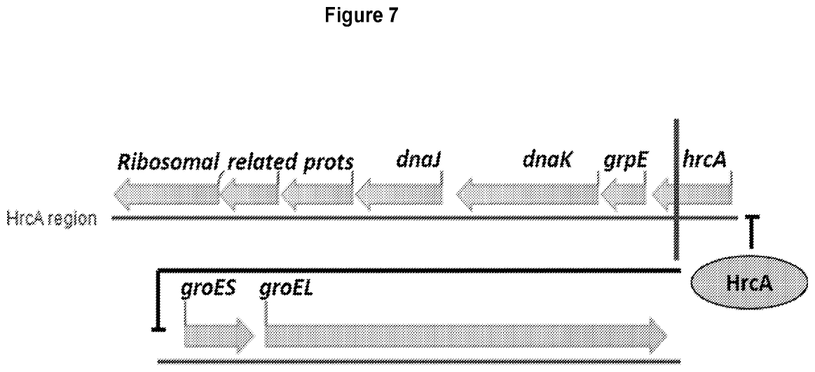

HrcA-HrcA has previously been described in B. subtilis as the transcriptional repressor of the class I heat-shock genes, the HrcA regulon (5). The HrcA regulon consists of just two operons, the heptacistronic dnak operon and the bicistronic groE (FIG. 7). Both operons encode for molecular chaperons and respond to heat stress. HrcA binds to the inverted repeats located in both operons and known as CIRCE (Controlling inverted repeat of chaperon expression) element. Dnak, DnaJ, GrpE and GroES-GroEL are chaperons over-expressed in response to both cold and high temperature treatments (Schumann, W. 2003). A B. subtilis hrcA knockout causes high constitutive expression of both class I heat-shock operons in the absence of heat-shock. In addition, deletion of hrcA in B. subtilis helps the strain to resume growth faster after a temperature change (heat or cold shock). Accordingly, HrcA over-expression has the opposite effect and cells are less capable to respond to temperature shifts (6).

Genome analysis of cold adapted derivatives identified mutations affecting the hrcA orf. Those SNPs corresponded in most cases to stop codons, and only in one of the mutant strains we identified an amino acid change Pro314 to Thr. Most likely all those mutations render an inactive repressor, which will consequently allow overexpression of the two operons in the regulon.

### CONCLUSION

To summarize, strains developed from B. paralicheniformis (DSM31110) with increased growth rates and higher biomass yields at different temperatures were developed following an adaptive laboratory evolution campaign. 14 different derivatives were selected, and their genomes sequenced to identify the acquired genotypic changes. Derivative strains were characterized physiologically and tested for performance in plant growth experiments.

Based on our results, specific mechanisms to explain the observed phenotypic differences with the parental strain is presented.

In addition, these evolution experiments have supplied/contributed with new Bacillus strains showing improved properties in plant growth promotion.

In a preferred aspect, the term bioF gene as used herein is intended to mean the BioF region 8-amino-7-oxononanoate synthase (EC_2.3.1.47).

In yet a preferred aspect, the term hrcA gene as used herein is intended to mean the Heat-inducible transcription repressor HrcA.

### DEPOSITS AND EXPERT SOLUTION

The applicant requests that a sample of the deposited microorganisms stated below may only be made available to an expert, until the date on which the patent is granted.

The applicant deposited Bacillus paralicheniformis parent strain was deposited as DSM31110 on May 8, 2019 at Leibniz Institute DSMZ—German Collection of Microorganisms and Cell Cultures, Inhoffenstr. 7B, D-38124 Braunschweig.

The applicant deposited the strains derived from the parent strain on Aug. 14, 2019 at Leibniz Institute DSMZ—German Collection of Microorganisms and Cell Cultures, Inhoffenstr. 7B, D-38124 Braunschweig as:

$$\begin{matrix}Bacillus paralicheniformis CHCC 32530 = DSM 33238 \\ Bacillus paralicheniformis CHCC 36494 = DSM 33239 \\ Bacillus paralicheniformis CHCC 36496 = DSM 33240 \\ Bacillus paralicheniformis CHCC 36751 = DSM 33241 \\ Bacillus paralicheniformis CHCC 36753 = DSM 33242 \\ Bacillus paralicheniformis CHCC 36754 = DSM 33243 \\ Bacillus paralicheniformis CHCC 36755 = DSM 33244\end{matrix}$$

The deposits were made according to the Budapest treaty on the international recognition of the deposit of microorganisms for the purposes of patent procedure.

### SEQUENCES

Forming part of present description is the sequence listing attached hereto.

As specified therein, the sequences
SEQ ID NO: 1 defines the hrcA nucleotide sequence
SEQ ID NO: 2 defines the HrcA protein sequence
SEQ ID NO: 3 defines the bioF nucleotide sequence
SEQ ID NO: 4 defines the BioF protein sequence.

1. A kit comprising:
(a) a Bacillus paralicheniformis having a mutation in its closest ortholog of the bioF gene found in the strain deposited at Leibniz Institute DSMZ—German Collection of Microorganisms and Cell Cultures, Inhoffenstr. 7B, D-38124 Braunschweig as accession number DSM 31110 (B. paralicheniformis DSM 31110);
(b) instructions for applying said Bacillus paralicheniformis to a fertilizer and/or a plant seed and/or a plant growth medium and/or a plant.

2. The kit of claim 1, wherein said Bacillus paralicheniformis has a genome that is at least 99% identical to the genome of B. paralicheniformis DSM 31110.

3. The kit of claim 1, wherein said closest ortholog is at least 95% identical to SEQ ID NO: 3.

4. The kit of claim 1, wherein said closet ortholog encodes a polypeptide that is at least 95% identical to SEQ ID NO: 4.

5. The kit of claim 1, wherein said mutation is a missense mutation.

6. The kit of claim 1, wherein said mutation is a missense mutation that produces an amino acid substitution at a position corresponding to Arg37, Ala64, Ala244, Asp300 or Pro314 of SEQ ID NO: 4.

7. The kit of claim 1, wherein said mutation is a missense mutation that produces an amino acid substitution, relative to SEQ ID NO: 4, selected from Arg37Trp, Ala64Val, Ala244Val, Asp300Gly and Pro314Ser.

8. The kit of claim 1, wherein said Bacillus paralicheniformis is derived from B. paralicheniformis DSM 31110.

9. The kit of claim 1, wherein said Bacillus paralicheniformis is selected from DSM 33239, DSM 33240, DSM 33243 or DSM 33244.

10. The kit of claim 1, wherein said Bacillus paralicheniformis is present in a composition comprising about 1×10² to about 1×10⁹ colony forming units of said Bacillus paralicheniformis per gram of said composition.

11. A method comprising applying a Bacillus paralicheniformis to a fertilizer and/or a plant seed and/or a plant growth medium and/or a plant, said Bacillus paralicheniformis having a mutation in its closest ortholog of the bioF gene found in the strain deposited at Leibniz Institute DSMZ—German Collection of Microorganisms and Cell Cultures, Inhoffenstr. 7B, D-38124 Braunschweig as accession number DSM 31110 (B. paralicheniformis DSM 31110).

12. The method of claim 11, wherein said Bacillus paralicheniformis has a genome that is at least 99% identical to the genome of B. paralicheniformis DSM 31110.

13. The method of claim 11, wherein said closest ortholog is at least 95% identical to SEQ ID NO: 3.

14. The method of claim 11, wherein said closet ortholog encodes a polypeptide that is at least 95% identical to SEQ ID NO: 4.

15. The method of claim 11, wherein said mutation is a missense mutation.

16. The method of claim 11, wherein said mutation is a missense mutation that produces an amino acid substitution at a position corresponding to Arg37, Ala64, Ala244, Asp300 or Pro314 of SEQ ID NO: 4.

17. The method of claim 11, wherein said mutation is a missense mutation that produces an amino acid substitution, relative to SEQ ID NO: 4, selected from Arg37Trp, Ala64Val, Ala244Val, Asp300Gly and Pro314Ser.

18. The method of claim 11, wherein said Bacillus paralicheniformis is derived from B. paralicheniformis DSM 31110.

19. The method of claim 11, wherein the Bacillus paralicheniformis is selected from DSM 33239, DSM 33240, DSM 33243 or DSM 33244.

20. A method comprising:
(a) providing a plurality of Bacillus candidate strains;
(b) detecting, in each Bacillus candidate strain, the presence or absence of:
i. a mutation in the closest ortholog of the hrcA gene of DSM 31110, optionally a loss-of-function mutation, optionally a nonsense mutation that introduces a premature stop codon; and/or
ii. a mutation in the closest ortholog of the bioF gene of DSM 31110, optionally a missense mutation, optionally missense mutation that produces an amino acid substitution at a position corresponding to Arg37, Ala64, Ala244, Asp300 or Pro314 of SEQ ID NO: 4, optionally a missense mutation that produces an amino acid substitution, relative to SEQ ID NO: 4, selected from Arg37Trp, Ala64Val, Ala244Val, Asp300Gly and Pro314Ser;
(c) selecting a Bacillus candidate strain comprising the mutation of step (b)(i) and/or step (b)(ii).
